(19) United States
(12) Patent Application Publication
(10) Pub. No.: US 2006/0241976 A1
(43) Pub. Date: Oct. 26, 2006

(54) System and method for determining CPT codes

(76) Inventor: Thomas W. Huth, Richmond, IN (US)

Correspondence Address: BAKER & DANIELS LLP, 300 NORTH MERIDIAN STREET, SUITE 2700, INDIANAPOLIS, IN, 46204, US

(21) Appl. No.: 11/115,057

(22) Filed: Apr. 26, 2005

(51) Int. Cl. G06F 19/00; A61B 5/00

(52) U.S. Cl. 705/3; 600/300

(57) ABSTRACT

A system for determining an applicable CPT code for the patient encounter. The system, in one form, includes a paper form defining a history section, an examination section and a complexity section. Each of the history and examination sections includes a plurality of point indicators, each of which is associated with a characteristic relating to a corresponding one of the sections. Each of the point indicators is marked during the patient encounter when the associated characteristic is applicable to the patient. Each of the history and examination sections also includes a section score calculator, which records a section tally of the marked point indicators and directs the conversion of the section tally to a section score. A final code calculator records the section score for each of the history and examination sections and a section score for the complexity section, and computes a final CPT code from the sections scores.

### BACKGROUND OF THE INVENTION

1. Field of the Invention

The present invention relates to systems for determining the applicable Evaluation and Management CPT Code for a health care encounter and for properly coordinating the associated documentation.

2. Description of the Related Art

Current Procedural Terminology (CPT) is a standardized coding system for identifying and categorizing the complexity and value of medical services. This coding system was originally developed by the American Medical Association in the 1960s. It was subsequently adopted by virtually all public and private health insurance payors in the United States for use by health care providers for the purpose of reporting claims for health care service reimbursement. Consequently, it is today the industry standard for the coding of medical services for all purposes.

The CPT system encompasses thousands of codes pertaining to the classification of virtually any health care service. The most commonly used, and among the most complex, are the Evaluation and Management (E & M) CPT codes. These codify the basic health care encounter in which a patient consults a practitioner for the evaluation and management of one or more acute or chronic health problems. There are more than one hundred E & M codes for a health care provider to choose from, the proper code for any particular encounter depending upon a large number of factors. Such factors may include: the type of practice; the place of service; whether the patient is new to the practice; the number, complexity, and severity of complaints and diagnoses; the number and types of items of medical history reviewed; the number and types of items of physical examination undertaken; the risks associated with various tests or treatments recommended; and many other factors. Regulators and payors expect that a health care provider will correctly determine the applicable E & M code for each patient encounter based upon a proper weighing of the various factors, and that the documentation in the patient's health record generated by the encounter will, upon auditing, support the appropriately chosen E & M code. Federal and State laws provide for sanctions against those health care providers who fail to properly report and document claims for health care services. These include not only sanctions for “overcoding” levels of service higher than can be supported by the documentation, but also “undercoding” levels of service lower than can be supported. “Undercoding” is regarded by the law to be an illegal kickback to a consumer so benefited. Auditing of coding compliance is increasingly a focus of efforts by regulators to reduce fraud, waste and abuse in the financing of the health care system. Nevertheless, the conceptual framework of the coding guidelines, and the practical steps necessary to properly code and document health care encounters, are unintuitive and therefore are commonly poorly understood by practitioners.

In the usual health care encounter, a patient is first evaluated and managed by a practitioner, and then a standardized billing form indicating the selected E & M code is completed. The criteriae for choosing among particular E & M codes are sufficiently complex and confusing that the typical practitioner resorts to “guesstimating” a code based on “feel” or simplified rules of thumb. The documentation for the encounter is almost always generated after assigning a CPT code so that other processing of the patient encounter can occur without awaiting completion of the documentation. Sometimes the documentation process is initiated immediately post-encounter, but often it is done at a more convenient later moment (such as at the end of the day) because it is time-consuming and slows patient flow if attended to immediately. Many practitioners dictate documentation, so that the transcription and review of the document may further delay finalization of the encounter, often by several days.

Several opportunities for error are thereby introduced into the health care encounter coding-and-documentation process. Practitioners are frequently uncertain how to apply the coding criteriae, and they are therefore uncertain how to coordinate the supporting documentation. In addition, sufficient time may have elapsed between an encounter and the documenting of it that the practitioner may be uncertain of the specifics of the encounter. The details of an encounter are even less well recollected when final transcribed documentation is eventually reviewed and finalized. Even if recollected accurately, the repetitious reconstruction of encounter details in the practitioner's mind at each step in the process is a taxing mental exercise, leading to fatigue and a tendency to underdocument.

A variety of methods have been developed to address one or more of the problems practitioners face in properly assigning E & M CPT codes and in properly coordinating the documentation. The most comprehensive are electronic medical record systems that require the practitioner to enter information into a computerized database during the patient encounter. The system automatically produces not only the encounter documentation but also the properly coded billing form, a recommended E & M code having been calculated via software algorithms but requiring final authorization by the practitioner. Several important objections to such an electronic system are frequently raised, however. These include such drawbacks as: a requisite large capital investment in hardware and software systems; costly system maintenance and upgrades; incompatibilities between an electronic system's logic flow and the thought process flow of the human practitioner; and the intrusion of technology into the human interaction between the patient and the practitioner.

Less comprehensive systems also exist to assist the practitioner in determining the applicable E & M CPT code. Some are desktop or handheld computer based software systems that produce a code recommendation after the practitioner checks off applicable attributes from lists of CPT code determination factors. Non-electronic slide rule-like E & M CPT code calculators and paper-based charts of CPT code factors are also available. Such tools reduce the guess-work in determining the CPT code at the conclusion of the patient encounter. However, they introduce an additional time-consuming step into the already compressed patient encounter. They also do little to improve the coordination of documentation and thus have a less-than-optimal impact on compliance with coding guidelines.

Other methods have been devised to streamline the documentation process. For example, paper-based standardized forms exist that allow the documentation of an encounter via check-boxes and fill-in blanks. These provide no means to calculate an applicable CPT code.

Accordingly, there is a need for an efficient, accurate, intuitive, and low cost system that can be used during a health care encounter to conveniently collect relevant information, to organize that information in a manner useful for calculating an E & M CPT code, to quickly and easily calculate the applicable CPT code from the organized information, and to serve as the medium for initiating properly coordinated documentation of the encounter.

### SUMMARY OF THE INVENTION

The present invention provides a system for use by a patient caregiver during a patient encounter to determine an applicable CPT code for the patient encounter. The system, in one form, includes a paper form defining a history section, an examination section and a complexity section. Each of the history and examination sections includes a plurality of point indicators, each of which is associated with a characteristic relating to a corresponding one of the sections. Each of the point indicators is marked during the patient encounter when the associated characteristic is applicable to the patient. Each of the history and examination sections also includes a section score calculator, which records a section tally of the marked point indicators and directs the conversion of the section tally to a section score. A final code calculator records the section score for each of the history and examination sections and a section score for the complexity section, and computes a final CPT code from the sections scores.

In another form, the system includes a paper form including at least one section. Each of the at least one sections includes a plurality of point indicators, each of which is associated with a characteristic relating to a corresponding one of the at least one section. A section score calculator is associated with each of the at least one sections. Each one of the plurality of point indicators in which the associated characteristic is applicable to the patient encounter receives a mark. The section score calculator records a section tally of the marks and converts the section tally to a section score.

The present invention also provides a method for use by a patient caregiver during a patient encounter to determine and document a CPT code applicable to the patient encounter. The method includes the steps on a paper form having a history section, an examination section and a complexity section, each of the sections including a plurality of point indicators, each of the point indicators being associated with a characteristic relating to a corresponding one of the sections; during the patient encounter, marking each one of the point indicators in which the associated characteristic applies to the patient encounter; calculating a section score for each of the sections by tallying the marked point indicators in each of the sections and using a section score calculator provided in each of the sections to convert the tally of marked point indicators in each section to a section score; determining the CPT code from the section score for each of the sections.

### BRIEF DESCRIPTION OF THE DRAWINGS

The above-mentioned and other features and objects of this invention, and the manner of attaining them, will become more apparent and the invention itself will be better understood by reference to the following description of embodiments of the invention taken in conjunction with the accompanying drawings, wherein:

FIG. 1 illustrates a first page of a paper system for determining applicable CPT codes according to one embodiment of the invention;

FIG. 2 illustrates a second page of the system shown in FIG. 1;

FIG. 3 is an exploded view of a subsection of the first page shown in FIG. 1;

FIG. 4 is an exploded view of another subsection of the first page shown in FIG. 1;

FIG. 5 is an exploded view of another subsection of the first page shown in FIG. 1;

FIG. 6 is an exploded view of a section of the second page shown in FIG. 2;

FIG. 7 is an exploded view of another section of the second page shown in FIG. 2

FIG. 8 is an example of the use of the first page of the paper system of FIGS. 1 and 2;

FIG. 9 is an example of the use of the second page of the paper system of FIGS. 1 and 2;

FIG. 10 is an example of the use of the first page of a paper system according to another embodiment of the invention; and

FIG. 11 is an example of the use of the second page of the paper system of FIG. 10.

### DETAILED DESCRIPTION

The embodiments hereinafter disclosed are not intended to be exhaustive or limit the invention to the precise forms disclosed in the following description. Rather the embodiments are chosen and described so that others skilled in the art may utilize its teachings.

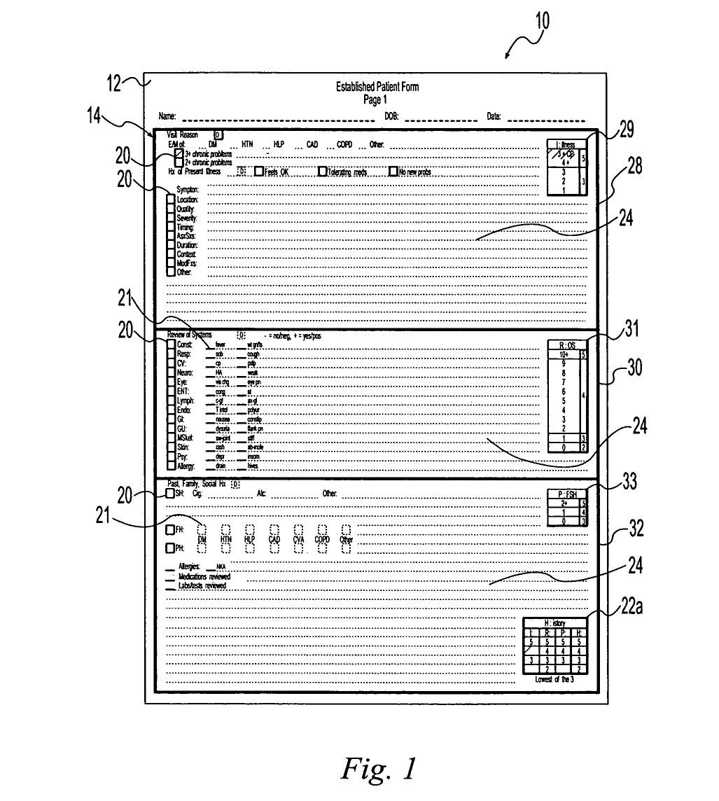

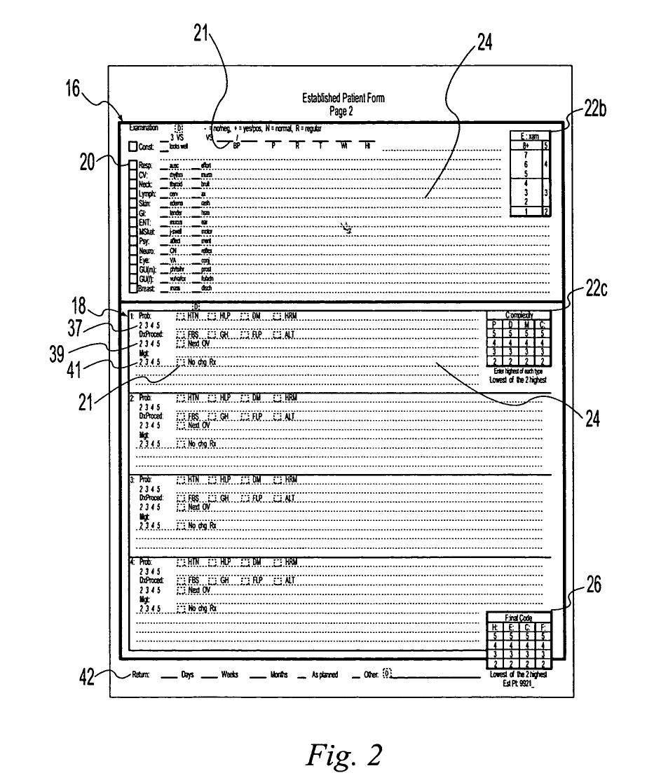

FIGS. 1 and 2 illustrate a tool or system, in accordance with the present invention, for determining the applicable CPT code for a patient encounter. As shown in FIGS. 1 and 2, the tool comprises a paper form 10, which may be conveniently and efficiently completed by a patient caregiver during a patient encounter. Although the present invention is embodied as a paper form in the Figures, it is contemplated that the form could be embodied in a digital format for completion using a desktop computer, laptop computer, handheld computer or similar electronic device.

As illustrated in FIGS. 1 and 2, form 10 generally includes patient demographic area 12 and three main patient encounter sections, including: history section 14, examination section 16 and complexity (Assessment/Plan) section 18. Patient demographic area 12 includes labeled blanks in which the patient's demographic information, such as name, date of birth (“DOB”), and date of patient encounter (“Date”), may be written. Patient demographic area 12 may be adapted to include additional or alternative patient information, such as social security number and insurance information.

Each of main patient encounter sections 14, 16, 18 includes a plurality of point indicators 20. Point indicators 20 are associated with, and adjacent to, an abbreviation of a characteristic related to the section within which the point indicator is located. Some point indicators 20 are also associated with one or more subindicators 21, each of which are associated with, and adjacent to, an abbreviation of a characteristic related to the section within which the subindicator is located. As is presented in further detail below, the characteristics may be any feature, fact or action relevant to the patient encounter and usually include those most commonly encountered in the particular patient care practice. For example, the characteristics may include systems of the body, symptoms, diagnostic procedures, diseases and social habits. Point indicators 20 and subindicators 21 are adapted to be quickly marked by the patient caregiver when the associated characteristic applies to the patient encounter. In form 10, point indicators 20 are in the form of solid lined boxes, while subindicators 21 are either in the form of blank lines or broken-lined boxes. However, point indicators 20 and subindicators 21 may be in any form suitable for quick marking, including check boxes, circles, or blanks. Subindicators 21 are, preferably, in a form distinguishable from indicators 20. Point indicators 20 may simply consist of the abbreviated characteristic itself which can be quickly circled or highlighted.

Each of main patient encounter sections 14, 16, 18 also includes a notation area 24 in which additional information regarding the patient encounter may be written. A section score calculator is associated with each of main patient encounter sections 14, 16, 18. More particularly, history section 14 includes history section score calculator 22a, examination section 16 includes examination section score calculator 22b, and complexity section 18 includes complexity section score calculator 22c. Section score calculators 22a-c are adapted such that a tally of the point indicators marked during the encounter in each of sections 14, 16, 18 during the patient encounter may be easily recorded on calculators 22a-c, respectively. Each of section score calculators 22a-c is also adapted to direct the conversion of each of the point indicator tallies to a section score, the process of which is discussed in further detail below. Form 10 also includes final code calculator 26, in which each of the section scores for sections 14, 16, 18 may be recorded. Final code calculator 26 is adapted to direct the conversion of the section scores to a final CPT code, the process of which is discussed in further detail below.

Referring back to FIGS. 1 and 2, paper form 10 is specifically designed for use in a family practice/internal medicine type setting and for evaluation of an established patient. However, as is discussed in further detail below, a similar form may be adapted in accordance with the present invention for use in other patient care practices and treatment settings and use with patients other than established patients.

Referring now to FIGS. 1-7, each of the sections of form 10 will now be discussed in more detail. Referring first to FIG. 1, history section 14 is divided into three subsections, illness subsection 28, review of systems (“ROS”) subsection 30, and past-family-social history (“PFS”) subsection 32. A history subsection score calculator 29, 31, 33 is associated with subsections 28, 30, 32, respectively.

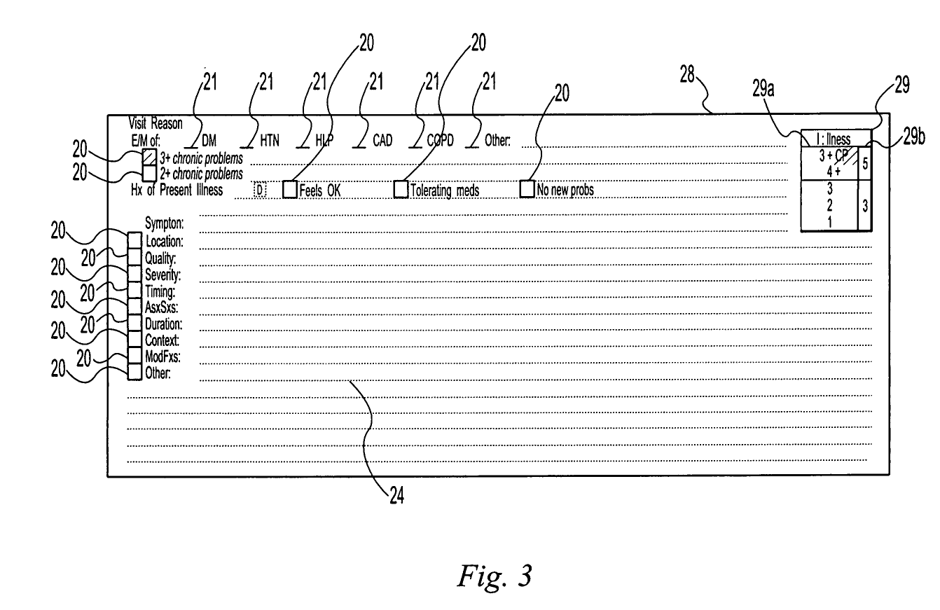

Turning to FIG. 3, illness subsection 28 of history section 14 relates to the evaluation of the reasons for the patient's visit and the history and symptoms of the present illness/complaint for which the patient is being seen. Point indicators 20 and subindicators 21 are associated with characteristics frequently discovered in the evaluation of illnesses commonly addressed in a family practice/internal medicine environment. Many of such characteristics are represented in illness subsection 28 in abbreviated form, the meaning of each may be found in Table I. Illness subsection 28 need not be restricted to those characteristics shown in FIG. 3 and listed in Table I. Rather, illness subsection 28 may be customized to include characteristics frequently encountered in a particular patient care practice. Illness subsection 28 also includes illness subsection code calculator 29, which includes a point indicator tally column 29a and an illness subsection score column 29b. The use of illness subsection score calculator 29 is discussed in detail below.

TABLE I
Illness Subsection

| Abbreviation | Meaning |
|---|---|
| E/M | evaluation and management |
| DM | diabetes mellitus |
| HTN | hypertension |
| HLP | hyperlipidemia |
| CAD | coronary artery disease |
| COPD | chronic obstructive pulmonary disease |
| Hx | history |
| Feels OK | feels okay |
| Tolerating meds | tolerating medications |
| No new probs | no new problems |
| AsxSxs | associated symptoms |
| ModFxs | modifying factors |

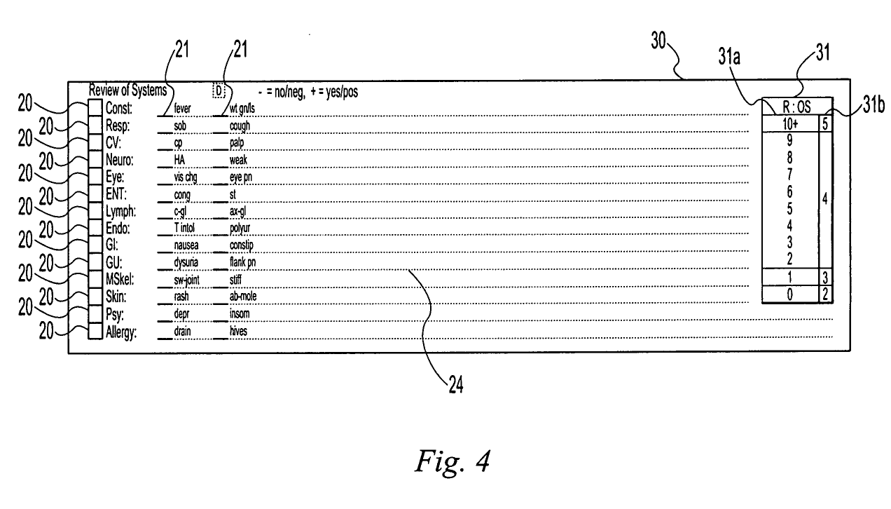

Referring to FIG. 4, ROS subsection 30 relates to the systems of the patient's body which are being reviewed or examined during the patient encounter. The systems commonly encountered during a typical patient encounter are associated with point indicators 20 in ROS subsection 30. These systems are represented in abbreviated form, the meaning of which may be found in Table II. Subindicators 21 in ROS subsection 30 are associated with abbreviations of symptoms commonly derived from the systems associated with point indicators 20 in ROS subsection 30. The meaning of these abbreviations may also be found in Table II. Again, the characteristics associated with point indicators 20 and subindicators 21 may be modified to include those often encountered in a particular patient care practice. As noted above, ROS subsection 30 includes ROS subsection code calculator 31, which includes a point indicator tally column 31a and an ROS subsection score column 31b. The use of ROS subsection code calculator 31 is discussed in detail below.

TABLE II
Review of Systems Subsection

| System | | Symptoms | | | |
|---|---|---|---|---|---|
| Abbreviation | Meaning | Abbreviation | Meaning | Abbreviation | Meaning |
| Const: | Constitutional | fever | fever | wt gn/ls | weight gain/loss |
| Resp: | Respiratory | sob | shortness of breath | cough | cough |
| CV: | Cardiovascular | cp | chest pain | palp | palpitations |
| Neuro: | Neurological | HA | headache | weak | weakness |
| Eye: | Eye | vis chg | vision change | eye pn | eye pain |
| ENT: | Ear, Nose, Throat | cong | congestion | st | sore throat |
| Lymph: | Lymphatic | c-gl | cervical glands | ax-gl | axillary glands |
| Endo: | Endocrine | T intol | temperature intolerance | polyur | polyuria |
| GI: | Gastrointestinal | nausea | nausea | constip | constipation |
| GU: | Genitourinary | dysuria | dysuria | flank pn | flank pain |
| MSkel: | Musculoskeletal | sw-joint | swollen joints | stiff | stiffness |
| Skin: | Skin | rash | rash | ab-mole | abnormal moles |
| Psy: | Psychological | depr | depression | insom | insomnia |
| Allergy: | Allergy | drain | nasal drainage | hives | hives |

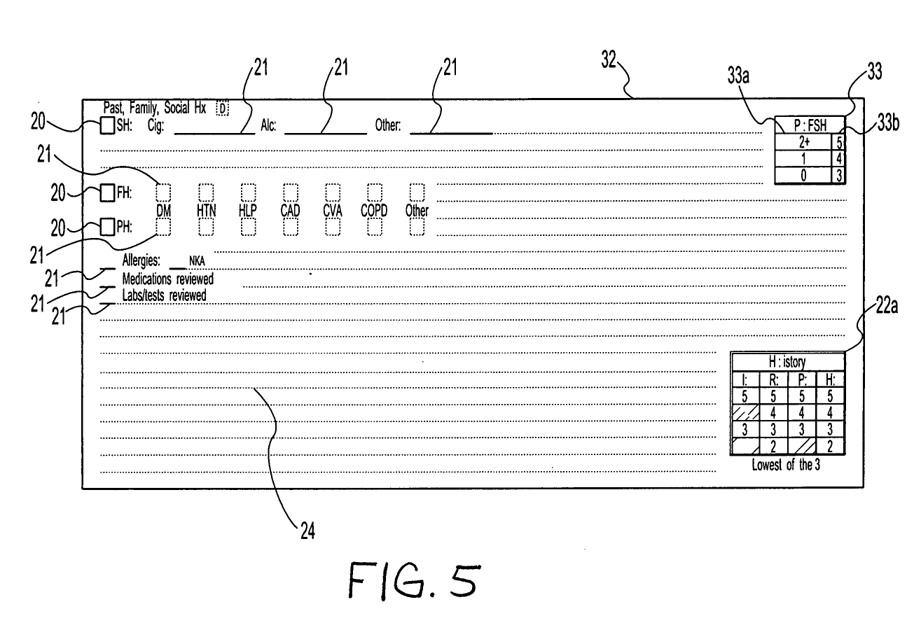

Referring to FIG. 5, PFS subsection 32 of history section 14 relates to the medical history of the patient and his/her family, as well as the social habits of the patient. Point indicators 20 and subindicators 21 of PFS subsection 32 are associated with abbreviations for characteristics commonly occurring in this area. The meaning of these abbreviations is noted in Table III. These characteristics may be changed to accommodate a particular patient practice.

TABLE III
Past, Family, Social History Subsection

| Abbreviation | Meaning |
|---|---|
| SH | social history |
| Cig | cigarettes |
| Alc | alcohol |
| FH | family history |
| PH | past medical history |
| NKA | no known allergies |
| DM | diabetes mellitus |
| HTN | hypertension |
| HLP | hyperlipidemia |
| CAD | coronary artery disease |
| COPD | chronic obstructive |
| | pulmonary disease |

History section 14 also includes history section score calculator 14a, which includes an illness column (labeled as “I:”) for recording the illness subsection score, an ROS column (labeled as “R:”) for recording the ROS subsection score, and a PFS column (labeled as “P:”) for recording the ROS subsection score. History section score calculator 14a is adapted to direct the calculation of a history section score from the recorded subsection score. The history section score calculator 14a also includes a history score column, labeled a “H:,” in which the calculated history section score may be recorded.

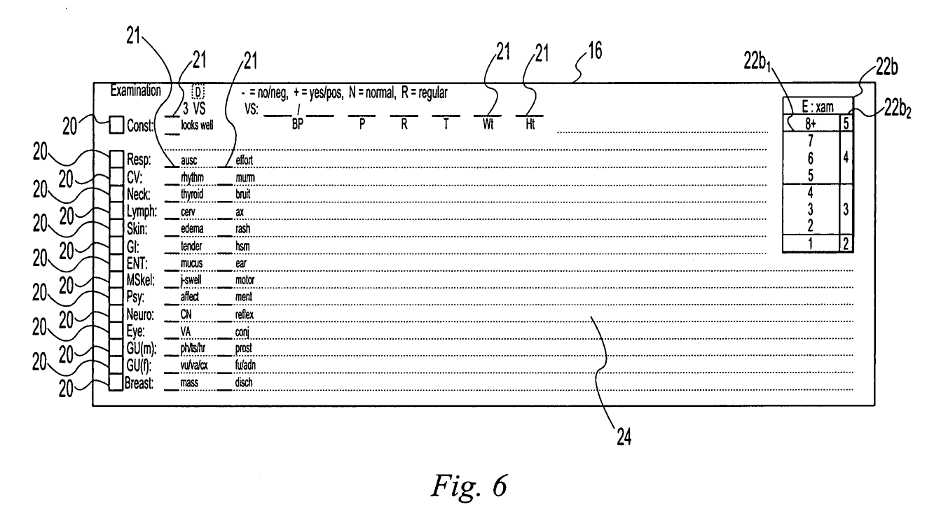

Referring now to FIG. 6, examination section 16 includes a plurality of point indicators 20, each associated with an abbreviation for a system of the body which is commonly examined during a patient encounter in the family practice/internal medicine setting. Each abbreviation is explained in Table IV. Each point indicator 20 and its related system is associated with two or more subindicators 21, each of which are associated with an abbreviation for a symptom relating to the system. An explanation of each of the abbreviations of these symptoms can be found in Table IV. The characteristics associated with indicators 20 and subindicators 21 may be modified to meet the activity of a particular practice. As noted above, examination section 16 includes examination section score calculator 22b, which includes a point indicator tally column 22b, and an examination score column 22b₂. The use of examination section score calculator 22b is discussed in detail below.

TABLE IV
Examination Section

| System | | Symptom | | | |
|---|---|---|---|---|---|
| Abbreviation | Meaning | Abbreviation | Meaning | Abbreviation | Meaning |
| Const: | Constitutional | VS | vital signs | effort | effort |
| Resp: | Respiratory | BP | blood pressure | murm | murmur |
| CV: | Cardiovascular | P | pulse | bruit | bruit |
| Neck: | Neck | R | respiratory rate | ax | axillary |
| Lymph: | Lymphatic | T | temperature | rash | rash |
| Skin: | Skin | Wt | weight | hsm | hepatosplenomegally |
| GI: | Gastrointestinal | Ht | height | ear | ears |
| ENT: | Ear, Nose, Throat | ausc | ausculation | motor | motor strength |
| MSkel: | Musculoskeletal | rhythm | rhythm | ment | mentation |
| Psy: | Psychological | thyroid | thyroid | reflex | reflexes |
| Neuro: | Neurological | cerv | cervical | conj | conjuntiva |
| Eye: | Eye | edema | edema | prost | prostate |
| GU(m): | Genitourinary (male) | tender | tender | fu/adn | fundus, adnexae |
| GU(f): | Genitourinary (female) | mucus | post-nasal mucus | disch | discharge |
| Breast: | Breast | j-swell | joint swelling | | |
| | | affect | affect | | |
| | | CN | cranial nerves | | |
| | | VA | gross visual acuity | | |
| | | ph/ts/hr | phallus, testes, | | |
| | | | hernia | | |
| | | vu/va/cx | vulva, vagina, cervix | | |
| | | mass | masses | | |

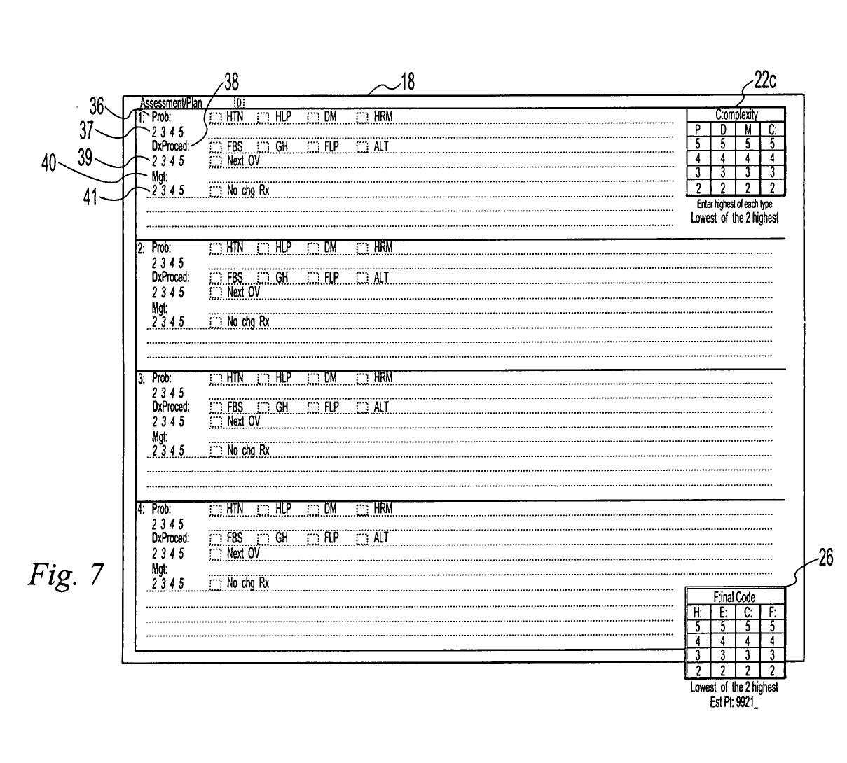

Referring to FIG. 7, complexity section 18 is divided into three subsections, problem (“Prob”) subsection 36, diagnostic procedures (“DX Proced”) subsection 38, and management (“Mgt”) subsection 40. Complexity section 18 includes repeating series of subsections 36, 38, 40 to accommodate multiple patient problems. Complexity section 18 and its subsections 36, 38, 40 include a plurality of subindicators associated with abbreviations for characteristics commonly useful in determining the complexity of the patient encounter. The characteristics include: symptoms/problems commonly complained of during a patient encounter, diagnostic procedures commonly assigned or reviewed during the patient encounter, and management strategies commonly used in treating the patient. The abbreviations in complexity section 18 and their meanings are listed in Table V. The characteristics of complexity section 18 may be modified and/or adapted to the specifications of a particular patient care practice. Each of subsections 36, 38, 40 includes corresponding subsection code calculators 37, 39, 41, respectively, in a subsection score may be recorded.

As discussed above, complexity section 18 includes complexity section score calculator 22c, which includes a problem column (labeled as “P”) for recording the problem subsection score, a diagnostic column (labeled as “D”) for recording the diagnostic subsection score, and a management column (labeled as “M”) for recording the management subsection or score. Complexity section score calculator 22c is adapted to direct the calculation of a complexity section score from the recorded subsection scores and includes a complexity score column (labeled as recorded “C”) in which the complexity score may be recorded.

TABLE V
Assessment/Plan Section

| Abbreviation | Meaning |
|---|---|
| Prob | problem |
| DxProced | diagnostic procedure |
| Mgt | management |
| HTN | hypertension |
| HLP | hyperlipidemia |
| DM | diabetes mellitus |
| HRM | high risk medication |
| FBS | fasting blood sugar |
| GH | glycohemoglobin |
| FLP | fasting lipid profile |
| ALT | alanine aminotransferase |
| Next OV | next office visit |
| No chg Rx | no change in medication |

It should be understood that the applicable CPT code for a particular patient encounter may vary depending on the status of the patient as either a new patient, a consultation patient or an established patient. Thus, the forms used for these two different types of patients may differ. FIGS. 1-7 illustrate a form 10 for use with an established patient. The form for use with a new/consultation patient (a patient that either is new, hasn't been seen by caregiver in a significant amount of time, or is just visiting for a single consultation), will look different and might involve different calculations. For instance, Tables VI, VII and VIII illustrate the history section score calculator, the examination score calculator and the final code calculator likely to exist in a new/consultation form.

TABLE VI
H:istory

| I: | R: | P: | H: |
|---|---|---|---|
| 5 | 5 | 5 | 5 |
| | 3 | 3 | 3 |
| 2 | 2 | 2 | 2 |
| | 1 | | 1 |

Lowest of the 3

TABLE VII
E:xam

| | |
|---|---|
| 8+ | 5 |
| 7 | |
| 6 | 3 |
| 5 | |
| 4 | |
| 3 | 2 |
| 2 | |
| 1 | 1 |

TABLE VIII
F:inal Code

| H: | E: | C: | F: |
|---|---|---|---|
| 5 | 5 | 5 | 5 |
| 4 | 4 | 4 | 4 |
| 3 | 3 | 3 | 3 |
| 2 | 2 | 2 | 2 |
| | 1 | | 1 |

Lowest of the three
New Pt: 9920_
Consult: 9924_

Referring now to FIGS. 1-7, the general procedure for using form 10 will now be described. When the patient arrives at the patient care facility (e.g., a physician's office), the status of the patient is determined as either a new patient, a consultation patient or an established patient and the appropriate form is selected according to the patient's status. Next, the patient's demographic information is entered into patient demographic section 12. This information may be simply handwritten on the paper form. Alternatively, the information may be keyed into a digital version of the form and then the paper form may be printed with the patient's demographics pre-printed on the resulting printed paper form. The demographics may be entered by the patient's caregiver or, more preferably, by the office staff in preparation for the patient encounter. The paper form is then made available to one or more patient caregivers for use during the patient encounter/examination.

During the patient encounter, the caregiver records information regarding the encounter on the form. More specifically, during a patient encounter a caregiver typically first inquires as to the reasons for the patient's visit. This information is entered into illness subsection 28 by first marking the subindicators 21 associated with the chronic problems the patient is experiencing. If the patient is experiencing chronic problems not listed in illness subsection 28, the caregiver marks the subindicator labeled “other” and records the other chronic problems in the notation area adjacent the “other” subindicator. The chronic problem subindicators are then counted and the appropriate point indicator associated with the total of marked subindicators is marked. Next, the symptoms of the patient's illness are discussed and recorded in the notation area 24 of illness section 28, and the point indicators related to the patient's symptoms are marked. The marked point indicators 20 in illness section 28 are counted and the total is marked in point tally column 29a of illness subsection score calculator 29. It should be understood that although the caregiver can count and record the total number of point indicators marked in the subsection quickly during the patient encounter, this task may be deferred to a later time and may be performed by staff/assistants.

Next, the patient caregiver typically makes a review or examination of the systems of the patient relevant to the patient's complaints and chronic problems. The patient caregiver marks subindicators 21 in the ROS subsection 30 related to the examination of these systems. Notes may be made regarding the review of systems in notation area 24 of ROS subsection 30. Next, the point indicators 20 relating to a marked subindicator and/or a system reviewed are checked. The checked point indicators are counted and the total is recorded in tally column 31a of ROS subsection calculator 31.

Next in the sequence of the form, the caregiver investigates the patient's past and family medical history and the patient's social habits. The information is recorded in notation area 24 of PFS subsection 32 and any applicable subindicators 21 are marked. Any point indicator 20 relating to a marked subindicator 21 is checked and the checked point indicators 20 of PFS subsection 32 are counted. The total number of checked point indicators in PFS subsection 32 are recorded in point tally column 33a of PFS subsection score calculator 33.

The patient caregiver then examines the patient and records the results of the examination in notation area 24 of examination section 16. The results may be marked on the subindicators 21 which relate to the examination findings. The caregiver then checks the point indicators relating to the systems of the body examined and relating to marked subindicators 21. The number of checked point indicators are counted and the total is recorded in point tally column 22b, of examination section score calculator 22.

Next, the caregiver evaluates each of the patient's problems, determines the diagnostic procedures necessary, and develops a treatment management program. In so doing, the caregiver marks the appropriate indicators 20 in complexity section 18 and/or records the information in notation area 24 of complexity section 18. The caregiver then estimates the level of risk with respect to the problem, diagnostic procedure, and management and marks the respective subsection calculators 37, 39, 41. Criteria for judging the risk level of each component of each problem addressed in the visit are determined using the CPT coding system and are not unique to the present invention. The criteria for judging the level of risk for each component is set forth below in Tables IX, X and XI.

TABLE IX
ASSIGNMENT OF RISK FOR PROBLEM

Level 2. Minimal risk:
One self-limited minor problem.
Level 3. Low risk:
The second (or more) self-limited minor problem;
one stable chronic illness;
or an acute uncomplicated illness or; injury requiring medical
intervention to avoid complications.
Level 4. Medium risk:
A chronic illness with mild exacerbation;
the second (or more) stable chronic illness;
an undiagnosed new problem with uncertain prognosis;
an acute illness with systemic symptoms;
or an acute injury with complications.
Level 5. High risk:
A chronic illness with severe exacerbation;
a life-threatening acute or chronic illness or injury;
or any problem accompanied by an abrupt change in neurological status.

TABLE X
ASSIGNMENT OF DIAGNOSTIC PROCEDURES FOR PROBLEM

Level 2. Minimal risk:
No tests, or tests by non-stress non-invasive and minimal risk means such as
venipuncture, standard X-ray, EKG or ultrasound.
Level 3. Low risk:
Low risk tests without significant physiologic stress, such as pulmonary function testing,
non-cardiovascular radiologic imaging with contrast, superficial biopsies or arterial
blood testing.
Level 4. Medium risk:
Medium risk tests, such as cardiovascular stress testing, gastrointestinal endoscopy, deep
needle biopsies, body cavity fluid sampling via needle puncture, or coronary
arteriography, in otherwise low risk patients. “Low risk patients,” generally, are those
lacking such risk factors as advanced age or unstable severe illnesses, among other
risk factors.
Level 5. High risk:
Medium risk tests in high risk patients;
or high risk tests, such as cardiac electrophysiologic testing, or heart or brain biopsy, in
any patient.

TABLE XI
ASSIGNMENT OF MANAGEMENT OPTIONS FOR PROBLEM

Level 2. Minimal risk:
No treatment;
or non-prescription, non-pharmacologic treatment such as rest, gargles,
dressings, and wraps.
Level 3. Low risk:
Low risk treatments such as non-prescription medications, physical
therapy or plain IV fluids without additives;
or minor surgery in a low risk patient.
Level 4. Medium risk:
Medium risk treatments such as prescription drug management, IV fluids
with additives, minor surgery in a high risk patient, major surgery in a
low risk patient, or radiation treatments.
Level 5. High risk:
High risk treatments such as major surgery in a high risk patient,
parenteral controlled substance management, toxic drug therapy
monitoring, or decision not to resuscitate.

If the caregiver would like the patient to return for a follow-up visit, he indicates the suggested follow-up period in follow-up section 42.

A section score is calculated for each section. This may be quickly performed during the patient encounter or at a later time. Turning to history section 14, an illness subsection score is first calculated using illness subsection score calculator 29. The illness score is determined by marking the number in score column 29b that corresponds to the point total marked in point tally column 29a. The illness subsection score is then carried forward to the illness column of history section score calculator 22a. The same process is repeated to determine the ROS and PFS subsection scores. The history subsection score is determined by following the brief instructions provided in history section score calculator 22a. For instance, in the established patient form shown in FIGS. 1-7, the instructions state “lowest of the 3.” Accordingly, the lowest of the three subsection scores (illness, ROS and PFS) is entered as the history score in the history score column of the history section score calculator 22a.

Turning to examination section score calculator 22b, the examination score is determined by marking the number in score column 22b₂ that corresponds to the point total in point tally column 22b₁. The marked number in score column 22b₂ is the examination score.

Turning to complexity section score calculator 22c, calculator 22c instructs the caregiver to enter a certain number in each of the “P,” “D,” and “M” columns, depending on the type of patient form. For instance, in the established patient form, complexity calculator 22c instructs the caregiver to enter the highest of each type or subsection. Accordingly, the highest of the problem subsection scores in problem subsection score calculators 37 is entered into column “P.” The highest of the diagnostic procedures scores in diagnostic subsection score calculators 39 is entered into column “D,” and the highest of the scores in management subsection score calculators 41 is entered into column “M.” Complexity score calculator 22c then instructs the user to select the lowest of the two highest subsection scores entered in columns “P,” “D” and “M”. This number is recorded in column “C” as the complexity score.

The history, examination and complexity scores are brought forward to final code calculator 26 and entered in columns “H:,” “E:,” and “C:,” respectively. The final code is calculated as instructed by final code calculator 26. For instance, final code calculator 26 of an established patient form instructs the user to take the lowest of the two highest section scores entered in columns “H:,” “E:,” and “C:.” This number is then inserted as the last digit of the CPT code, the first 4 digits of which correspond to the patient's status and are set forth under final code calculator 26. The form 10 may then be submitted to a billing function where the CPT code is entered and the supporting patient encounter information is transcribed.

### EXAMPLE I

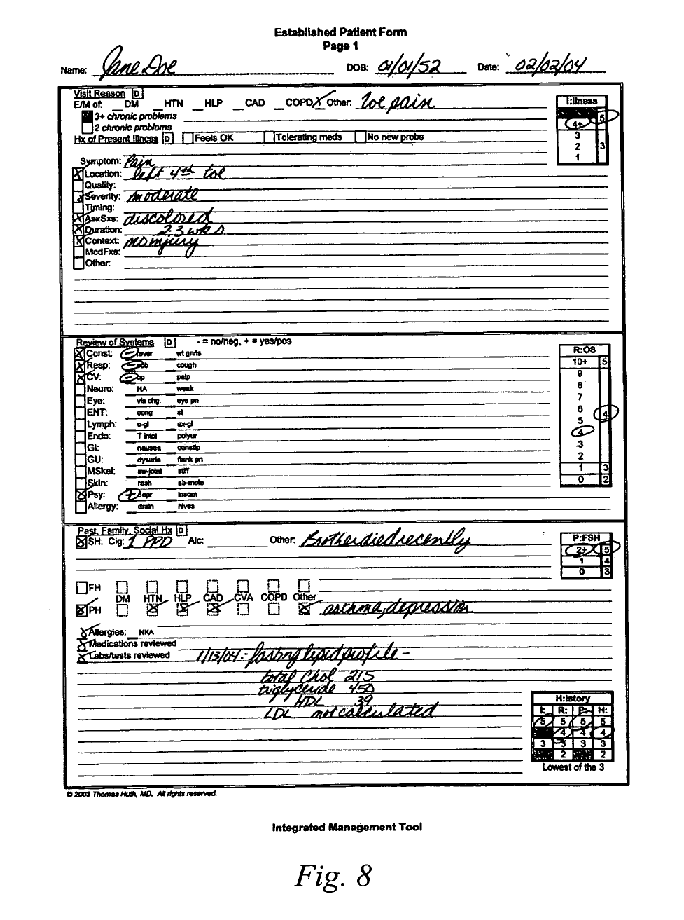

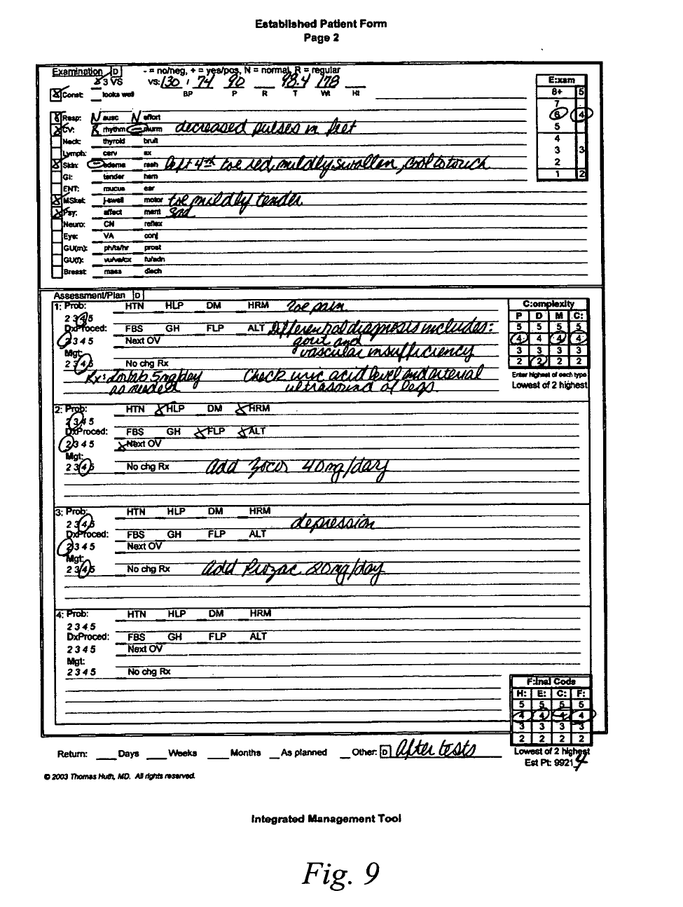

Referring now to FIGS. 8 and 9, an exemplary use of the form will now be described. The patient status in this example is an established patient and, therefore, the established patient form is selected. Turning to FIG. 8, the patient's demographic information, in this case the patient's name, date of birth, and date of visit, is recorded in the demographic section of the form. During the patient encounter, the caregiver determines that the patient, Jane Doe, has been experiencing moderate pain and discoloration in her left fourth toe for about two to three weeks. Accordingly, the appropriate point indicators relating to the evaluation of the toe pain symptoms are marked. More specifically, the caregiver records the location of the symptom as the left fourth toe and checks the point indicator relating to location. The caregiver also checks the point indicators relating to the severity, associated symptoms (AsxSxs) and duration, and records the severity of the pain as moderate, the associated symptom of discoloration and the duration as 2-3 weeks. The caregiver also checks the point indicator relating to context and notes that the patient has not sustained any injury to the affected toe. In this case, a total of five point indicators (location, severity, AsxSxs, duration and context) have been marked in the illness section. Accordingly, in the point tally column of the illness subsection score calculator, “4+” is circled as the total of the point indicators in the illness section. Using the illness subsection score calculator, this quickly yields an illness score of 5.

Referring to the ROS section, the caregiver inquires as to the condition of the patient's following systems: constitutional, respiratory, cardiovascular and psychological. For instance, in the review of the constitutional system, the caregiver finds no indications of a fever, but notes fatigue. This information is recorded by entering a negative sign in the fever subindicator and noting fatigue in the notation area. This review yields a check or mark in the point indicator related to the constitutional system. Similarly, in the respiratory system, the patient caregiver records a negative for shortness of breath and, thus, checks the point indicator related to the respiratory system. The patient caregiver also notes that the patient has had no signs of chest pain and, consequently, marks a negative sign next to the subindicator “cp.” This review earns a check in the point indicator next to the cardiovascular system. The patient caregiver further inquires about the psychological status of the patient and determines that the patient is experiencing depression. The patient caregiver accordingly marks a positive sign in the subindicator relating to depression. Next, the point indicators in the ROS subsection are tallied and a total number of checked point indicators is recorded in the point tally column of the ROS subsection calculator. In this case, this particular patient encounter yielded four marked point indicators in the ROS subsection and, thus number 4 is circled in the point tally column of the ROS subsection calculator. The ROS score calculator indicates that a tally of four marked indicators corresponds to an ROS subsection score of 4, and the 4 is circled in the score column.

Next, the caregiver reviews and investigates the past and family medical history of the patient and the patient's social habits. During this investigation the patient caregiver discovers that the patient smokes about one pack of cigarettes per day. This notation is made in the subindicator labeled “Cig” which earns a check in the point indicator box labeled “SH.” The caregiver further discovers that the patient's brother recently died and records this information in the notation area next to “SH.” The caregiver also discovers that the patient has a history of asthma, depression, hypertension (HTN), hyperlipidemia (HLP) and coronary artery disease (CAD), and checks the corresponding subindicators. This results in a check in the PH point indicator. The caregiver also notes that the patient has had allergies and reviews the patient's medication and laboratory tests. The caregiver notes the patient's current medications in the notation area, as well as the results from the patient's recent lab tests. The caregiver counts the number of point indicators marked in the PFS subsection and notes that the number of marked point indicators totals two. The caregiver circles the “2+” in the point tally column of the PFS subsection calculator. This point tally converts to a subsection score of five.

The illness, ROS and PFS subsection scores are then forwarded to the history section score calculator, wherein these scores are recorded in columns I:, R: and P:, respectively. As shown in the history section score calculator, the illness score was 5, the ROS score is 4 and the PFS score is 5. The history section score calculator instructs the caregiver to select the lowest of these three subsection scores. This yields a 4 as the history score, which is recorded in the history column of the history section score calculator.

Referring now to FIG. 9 and the examination section, the caregiver takes the vital signs of the patient and records them in the corresponding subindicators. This yields a check in the constitutional point indicator. The caregiver also examines the patient's respiratory system and notes a negative in both of the related subindicators. An examination is also made of the patient's cardiovascular system and notes regular (R) in the rhythm subindicator and a negative in the subindicator for murmurs. The caregiver also notes in the notation area that the patient has decreased pulses in the foot. These notations earn a check in the point indicator relating to cardiovascular (CV). The skin is examined and the skin point indicator is also checked. Notations regarding the skin examination are made in the notation area. The caregiver notes that the toe was red and swollen, and the foot was cool. A negative sign is inserted in the sub-indicator for edema. The patient caregiver examines the musculoskeletal system and finds that the fourth left toe is tender. This earns a checkmark in the musculoskeletal point indicator. Finally, an examination is made of the psychological system of the patient resulting in a notation in the notation area that the patient is experiencing sadness. This earns a check in the psychological point indicator. The caregiver counts the checked point indicators to yield a total of 6 checked point indicators. The examination section score calculator indicates that the 6 equals an examination score of 4 and the 4 is circled in the score column of examination score calculator.

Finally, an assessment of the complexity/risk of each of the patient's problems is made in the complexity section. First, the caregiver makes a notation of the toe pain. The caregiver, using his or her knowledge of the CPT coding criteria listed above, then estimates the complexity of the problem as a 4. The caregiver then diagnoses the problem as gout and vascular insufficiency. The caregiver rates the diagnosis procedures as a 2. The caregiver orders a check of the uric acid levels and an arterial ultrasound. The caregiver prescribes Lortab as needed and rates the management as a complexity 2. Next, the caregiver addresses the patient's hyperlipidemia (HLP) and high risk medication (HRM), and rates the problem as a 3. The caregiver orders a fasting lipid profile (FLP) and an alanine aminotransferase (ALT) next office visit. These diagnostic procedures are rated as a 2. The caregiver then adds Zocor 40 mg/day to the patient's prescribed medications and rates this management as a 4. Finally, the caregiver addresses the patient's depression. The caregiver prescribes Prozac for management of the depression. The caregiver rates the complexity of the problem as a 4, the complexity of the diagnosis procedures as a 2, and the complexity of the management of the problem as a 4.

The caregiver then refers to the complexity section score calculator and is instructed to enter the highest of each type of subsection. The highest score for the problem subsection is 4 and, therefore, a 4 is circled in the column labeled “P:.” The highest score for the diagnostic procedures is 2 and, therefore, a 2 is circled in the “D:” column. The highest score for the management subsection is 4 and, therefore, a 4 is circled in the “M:” column of the complexity section score calculator. The complexity score calculator then instructs the patient caregiver that the complexity score is equal to the lowest of the two highest subsection scores. The two highest subsection scores are the 4 for the problem and the 4 for the management. The lowest of these two highest scores is 4. Consequently, the 4 is circled as the complexity score in the complexity column of complexity section score calculator.

The caregiver then decides that the patient should return after the tests are complete. This is indicated in the patient follow-up section of the form.

Finally, the final code is determined by forwarding the section scores from the history section, examination section, and complexity section to the H:, E: and C: columns of the final code calculator. As shown in the final code calculator, the history score is 4, the examination score is 4 and the complexity score is 4. The final code calculator instructs the caregiver that the final code will be equal to the lowest of the two highest scores. The scores are 4 in all the sections and, thus, the lowest of the two highest scores is a 4. The 4 is then inserted as the final digit of the five-digit CPT code, as shown below the final code calculator.

As illustrated in this example, the CPT code may be quickly determined in real time during the patient encounter by the caregiver in the caregiver's normal course of recording information regarding the patient. As a result, the system of the present system reduces the uncertainties and inaccuracies resulting from the gap between the patient encounter and the conventional subsequent determination of CPT codes. This ultimately creates a more efficient and accurate coding and billing system, benefiting the patient and the physician, and provides more accurate data to the reimbursement payor.

### EXAMPLE II

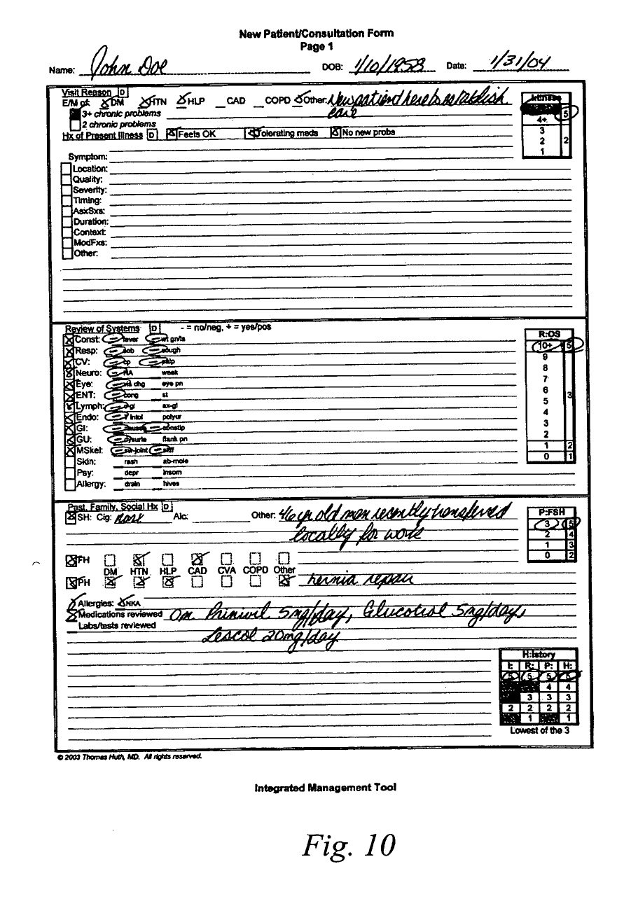

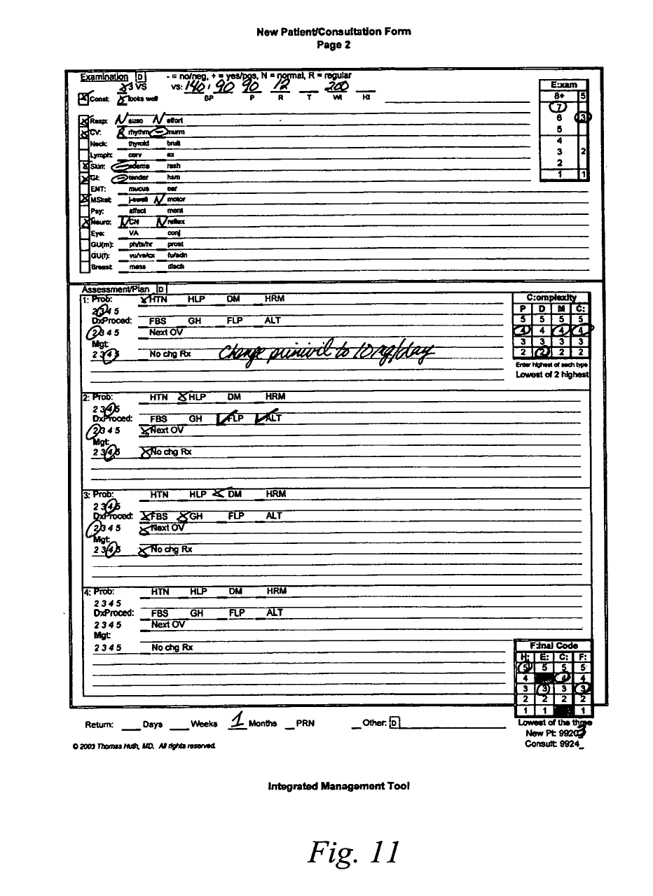

Referring now to FIGS. 10 and 11, another exemplary use of the present invention will be illustrated. In this example, John Doe has recently been transferred by his employer to the area and he needs to establish a new patient care relationship with a physician in the area. Because John Doe is a new patient, the New Patient Form is selected. As shown in FIG. 10, John Doe's name, date of birth and date of visit is entered in the patient demographic section of the New Patient Form. The physician or caregiver investigates John's present illnesses and determines that John Doe has diabetes mellitus (DM), hypertension (HTN) and hyperlipidemia (HLP). As a result, the caregiver checks the subindicators relating to “DM,” “HTN,” “HLP” and “Other.” Since John Doe has 3 or more chronic problems (i.e., DM, HTN, HLP), the point indicator indicating “3+” chronic problems is checked. Moving to the illness subsection score calculator, the “3+CP” is circled in the point tally column. The illness subsection score calculator indicates that 3+chronic problems yields an illness subsection score of 5.

Moving to the ROS section, the caregiver reviews the following systems: constitutional, respiratory, cardiovascular, neurological, eye, ear-nose-throat, lymphatic, endocrine, gastrointestinal, genitourinary and musculoskeletal. With respect to John's constitutional system, the caregiver notes a negative in the subindicators for fever and weight gain or loss, and checks the constitutional point indicator. The caregiver then notes that John has not been experiencing any shortness of breath or cough and, thus, marks a negative in these subindicators and checks the respiratory point indicator. Next, the caregiver investigates John's cardiovascular experiences and notes that John has not been experiencing any chest pain or palpitations. As a result, the caregiver marks a negative in the cp and palp subindicators and checks the cardiovascular point indicator. The caregiver determines that John has not had headaches and, thus, marks the HA subindicator as negative and checks the neurological point indicator. The caregiver then inquires at to the condition of John's eyes and finds that John has experienced no changes in vision. Consequently, the caregiver marks a negative in the subindicator for vision change and checks the point indicator for eye. In investigating John's ear-nose-throat history, the caregiver finds no recent congestion and marks a negative for the congestion subindicator. This earns a check in the point indicator relating to ENT. Next, the caregiver inquires about John's lymphatic system and finds no cervical gland problems. This earns a negative in the “c-gl” subindicator and a check in the lymphatic point indicator. The caregiver then notes a negative for temperature intolerance and checks the endocrine point indicator. The caregiver then finds no recent history of nausea or constipation and marks a negative in these subindicators. This yields a check in the gastrointestinal point indicator. John indicates no history of dysuria and, therefore, the caregiver marks a negative in the dysuria subindicator and checks the genitourinary point indicator. Finally, the caregiver finds that John has no recent history of joint swelling or stiffness. Consequently, the caregiver marks the subindicators negative and checks the point indicator for musculoskeletal. The caregiver then tallies all the checked point indicators and totals 11. The caregiver marks the “10+” in the point tally column of the ROS subsection calculator. The ROS score calculator indicates that a point tally of 10 or more yields an ROS subsection score of 5 and, thus, a 5 is circled in the ROS score column.

Next, the caregiver investigates John's past, family and social history. First, the caregiver discovers that John does not smoke and that he was recently transferred to the area for work. The caregiver notes this information in the notation area of the PFS section. The caregiver then finds that John has a family history of hypertension and coronary artery disease. The caregiver records this information by checking the appropriate subindicators, which yields a check in the Family History point indicator.

John's past medical history reveals that he has had hernia repair and has experienced diabetes, hypertension, hyperlipidemia, and coronary artery disease. Accordingly, the corresponding subindicators and the point indicator for past history are checked. The caregiver next finds that John has no known allergies and that he currently takes 5 mg/day of Prinivil, 5 mg/day of Glucotrol, and 20 mg/day of Lescol. This information is recorded in the notation area of the PFS subsection. The caregiver tallies the checked point indicators and totals 3. The caregiver circles the 3 in the point tally column of the PFS subsection score calculator, which indicates that the PFS section score is 5. The caregiver circles the 5 in the score section of the PFS subsection score calculator. The Illness, ROS, and History scores are then brought forward to the History score calculator and are entered into columns “I:,” “R:,” and “P:,” respectively. The History score calculator instructs the caregiver to take the lowest of these three subsection scores as the history score. Since all three subsections earned a 5, 5 is the lowest score and a 5 is circled in the History score column.

Turning now to FIG. 1, the caregiver examines John Doe and records the information in the Examination section. The caregiver starts by taking and recording John's vital signs, including blood pressure, pulse, respiration and weight. Since at least three vital signs were recorded, the “3vs” subindicator was checked. The caregiver also notes that John Doe looks well and records this by checking the corresponding subindicator. These examinations result in a check of the point indicator relating to the constitutional system. The caregiver examines John's respiratory system and notes that it is N (normal) for ausculation and effort. This examination yields a check in the point indicator for respiratory.

Referring still to FIG. 1, the caregiver examined John's cardiovascular system and found normal rhythm and no murmurs. As a result, the caregiver checked the point indicator for cardiovascular. The caregiver examined John's skin and found no signs of edema. Accordingly, the caregiver marked a negative in the edema subindicator and checked the point indicator relating to skin. The caregiver then examined John's gastrointestinal system and found no tenderness. The caregiver marks the gastrointestinal point indicator. Next, the caregiver examines John's musculoskeletal system and finds his motor strength to be normal. As a result, the caregiver notes an “N” for normal in the motor subindicator and checks the point indicator for the musculoskeletal system. Finally, the caregiver examines John's neurological system. Finally, the caregiver examines John's neurological system and finds that his cranial nerves and reflexes are normal. This examination results in a check in the point indicator for the neurological system. The caregiver tallies the checked point indicators and counts 7. The number 7 is marked in the point tally column of the Examination subsection score calculator, which indicates that a point tally of 7 yields a score of 3. As a result, the 3 is circled in the Examination score column.

Finally, an assessment of the complexity/risk of the patient's problems is performed and recorded in the complexity section. First, the caregiver addresses John's hypertension and changes his dosage of Prinivil from 5 mg/day to 10 mg/day. Using the CPT coding criteria described above, the caregiver assigns the problem as a risk of 4, the diagnostic procedure as a risk of 2 and the management as a risk of 4. The caregiver then addresses John's hyperlipidemia and assigns a follow-up office visit and makes no changes in medication. The caregiver assigns the problem as a risk of 4, the diagnostic procedure as a risk of 2, and a management risk score of 4. Finally, the caregiver assesses John's diabetes and orders diagnostic procedures: fasting blood sugar (FBS) and glycated hemoglobin (GB). The caregiver orders another office visit and makes no changes to the medications. The caregiver assigns the problem a risk of 4, the diagnostic procedures a risk of 2 and the management a risk of 4. Complexity score calculator instructs the caregiver to enter the highest risk score for each of Problem, Diagnostic Procedures, and Management in columns “P,” “D,” and “M,” respectively. Consequently, the caregiver enters a 4 in column P, a 2 in column D and a 4 in column M. The complexity score calculator then instructs the caregiver to enter the lowest of the two highest subsection scores as the Complexity section score. The two highest subsection scores are 4 for the Problem subsection and 4 for the Management subsection, and the lowest of these two is 4. Accordingly, a 4 is entered in the Complexity score column.

The History, Examination and Complexity scores are brought forward to the H:, E: and C: columns of the final code calculator. As illustrated, the History score is 5, the Examination score is 3, and the Complexity score is 4. The final code calculator instructs the caregiver that the final code will be equal to the lowest of the three. The lowest of the three section scores is 3 for the Examination section and, thus, a 3 is entered in the final score column. The 3 is then entered as the final digit of the new patient CPT code.

Although, the above examples and the FIGS. illustrate the tool of the present invention adapted for use in a family practice/internal medicine environment, the tool may be adapted for use in any particular practice. For instance, the tool may be adapted for use in an Ear Note Throat (ENT) practice. In this case, the characteristics listed/associated with the point indicators and subindicators might relate to symptoms and systems frequently encountered in an ENT practice. Such characteristics might include such systems and symptoms as: frontal sinus, ethnoid sinus, maxillary sinus, deviated septum, sinusitis, turbinates, tonsils, adenoids, ear, eustacian tube, eardrum, hearing loss, otitus, and other related characteristics. The tool may be similarly adapted for use in patient care practices such as pediatrics, cardiology, physical therapy and others.

While this invention has been described as having an exemplary design, the present invention may be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains.

What is claimed is:

1. A system for determining an applicable CPT code for a patient encounter, the system comprising:
a paper form including a history section, an examination section and a complexity section, each of said history and examination sections including a plurality of point indicators, each of said point indicators being associated with a characteristic relating to a corresponding one of said sections,
wherein each of said point indicators is marked during the patient encounter when said associated characteristic is applicable to the patient.

2. The system of claim 1 wherein each of said history and examination sections includes a section score calculator, said section score calculator records a section tally of said marked point indicators and directs the conversion of said section tally to a section score.

3. The system of claim 2 wherein said paper form further includes a final code calculator, said final code calculator records said section score for each of said history and examination sections and a section score for said complexity section, said final code calculator computes a final CPT code from said sections scores.

4. The system of claim 1 wherein said history section includes a first subsection, a second subsection, and a third subsection, each of said subsections includes a number of said plurality of point indicators in said history section, each of said point indicators of said number is associated with a characteristic relating to a corresponding one of said subsections, each of said subsections includes a subsection score calculator, said subsection score calculator records a subsection tally of said marked point indicators and converts said subsection tally to a subsection score, said section score calculator receives said subsection score to yield said section tally.

5. The system of claim 1 wherein said complexity section includes a first subsection, a second subsection, and a third subsection, each of said subsections includes a subsection score calculator, said subsection score calculator records a subsection score, said section score calculator receives said subsection score of each of said subsections and directs the calculation of a section score from said subsection scores.

6. The system of claim 1 wherein each of said sections includes a notation area for recording information relating to the patient encounter.

7. A method for determining an applicable CPT code using the system of claim 1.

8. A tool for use by a patient caregiver during a patient encounter to determining a CPT code applicable to the patient encounter, the tool comprising:
a paper form including at least one section, each of said at least one section including a plurality of point indicators, each of said point indicators being associated with a characteristic relating to a corresponding one of said at least one section, and a section score calculator associated with each of said at least one section,
wherein each one of said plurality of point indicators in which said associated characteristic is applicable to the patient encounter receives a mark, said section score calculator records a section tally of said marks and converts said section tally to a section score.

9. The tool of claim 8 wherein said paper form further includes a final code calculator, wherein said section score for each of said at least one section is recorded, said final code calculator computing a final CPT code from said sections scores.

10. The tool of claim 8 wherein one of said at least one section includes a first subsection, a second subsection, and a third subsection, each of said subsections includes a number of said plurality of point indicators in said one of said at least one section, each of said point indicators of said number are associated with a characteristic relating to a corresponding one of said subsections, each of said subsections includes a subsection score calculator, said subsection score calculator records a subsection tally of said marked point indicators and converts said subsection tally to a subsection score, said section score calculator receives said subsection score of each of said subsections to yield said section tally.

11. The tool of claim 8 wherein each of said sections includes a notation area for receiving information relating to the patient encounter.

12. A method for use by a patient caregiver during a patient encounter to determine and document a CPT code applicable to the patient encounter, the method comprising the steps of:
selecting a paper form having a history section, an examination section and a complexity section, each of the sections including a plurality of point indicators, each of the point indicators being associated with a characteristic relating to a corresponding one of the sections;
during the patient encounter, marking each one of the point indicators in which the associated characteristic applies to the patient encounter;
calculating a section score for each of the sections by tallying the marked point indicators in each of the sections and using a section score calculator provided in each of the sections to convert the tally of marked point indicators in each section to a section score;
determining the CPT code from the section score for each of the sections.

13. The method of claim 12 further comprising recording information relating to the patient encounter and the marked point indicators in a notation area provided in the paper form.

14. The method of claim 12 wherein the history section has a plurality of history subsections, each of the history subsections including a number of the plurality of point indicators in the history section, each of the point indicators of each history subsection is associated with a characteristic relating to a corresponding one of said history subsections, and wherein the step of calculating a section score for each of the sections further includes:
tallying the marked point indicators in each of the history subsections;
recording the tally of the marked point indicators of each of the history subsections in a history subsection score calculator provided in each of the history subsections;
using the history subsection score calculator in each of the history subsections to calculate a history subsection score for each of the history subsections; and
using the section score calculator to convert the subsection scores to a section score.

15. The method of claim 12 wherein the step of selecting a paper form includes determining the status of a patient as either an established patient, a new patient, or a consultation patient and selecting a paper form corresponding to the determined status.

16. The method of claim 12 wherein the CPT code comprises a final digit and wherein in said step of determining the CPT code from the section score for each of the sections includes selecting one of the section scores as a final digit of the CPT code.

17. The method of claim 16 wherein in the step of determining the CPT code from the section score for each of the sections a final code calculator provided on the form directs the selecting of one of the section scores as a final digit of the CPT code based on the status of the patient.

18. The method of claim 12 further comprises recording patient demographics in a patient demographic section provided on the paper form.

19. The method of claim 12 wherein the CPT code is documented into an accounting system.

20. The method of claim 19 wherein the information recorded on the form is entered into the accounting system to substantiate the CPT code.
